(19) United States
(12) Patent Application Publication
(10) Pub. No.: US 2024/0165113 A1
(43) Pub. Date: May 23, 2024

(54) METHODS OF TREATMENT FOR LEUKEMIA

(71) Applicant: DNA SEQ 2, Inc., San Diego, CA (US)

(72) Inventor: Janusz Sowadski, San Diego, CA (US)

(21) Appl. No.: 18/549,564

(22) PCT Filed: Apr. 8, 2022
(86) PCT No.: PCT/US2022/024047

§ 371 Date: Sep. 7, 2023

Related U.S. Application Data
(60) Provisional application No. 63/173,292, filed on Apr. 9, 2021.

(51) Int. Cl. A61K 31/505

(52) U.S. Cl. CPC A61K 31/505

(57) ABSTRACT

Provided herein are methods of use a cyclin dependent kinase (CDK) inhibitor for the treatment of leukemia. The CDK inhibitor described herein include NU6027. The methods described herein include methods of treatment of drug-resistant leukemia, such as leukemia presenting ABL1 mutations and chronic myelogenous leukemia (CML) comprising the administration of NU6027.

### CROSS-REFERENCE TO RELATED APPLICATION

This application claims the benefit of priority under 35 U.S.C. § 119(e) of U.S. Provisional Application No. 63/173,292 filed Apr. 9, 2021, the entire content of this application is incorporated herein by reference in its entirety.

### BACKGROUND OF THE INVENTION

### Field of the Invention

The present invention relates generally to the treatment of leukemia and more specifically to the treatment of drug-resistant leukemia.

### Background Information

Leukemia is a group of cancers that develops in the early blood-forming cells, that usually begins in the bone marrow and that results in high numbers of abnormal blood cells, which are not fully developed and called blasts or leukemia cells. Most often, leukemia is a cancer of the white blood cells, but some leukemias start in other blood cell types. There are several types of leukemia, which are divided based mainly on whether the leukemia is acute (fast growing) or chronic (slower growing), and whether it starts in myeloid cells or lymphoid cells. There are four main types of leukemia—acute lymphoblastic leukemia (ALL), acute myeloid leukemia (AML), chronic lymphocytic leukemia (CLL) and chronic myeloid leukemia (CML)—as well as a number of less common types. Leukemias and lymphomas both belong to a broader group of tumors that affect the blood, bone marrow, and lymphoid system, known as tumors of the hematopoietic and lymphoid tissues.

The exact cause of leukemia is usually unknown, with the combination of genetic factors and environmental (non-inherited) factors believed to play a role. Risk factors include smoking, ionizing radiation, some chemicals (such as benzene), prior chemotherapy, and Down syndrome. People with a family history of leukemia are also at higher risk. Different types of leukemia have different treatment options and outlooks. Treatment may involve some combination of chemotherapy, radiation therapy, targeted therapy, and bone marrow transplant, in addition to supportive care and palliative care as needed. The success of treatment depends on the type of leukemia and the age of the person.

CML was the first cancer to be linked to a clear genetic abnormality, the chromosomal translocation known as the Philadelphia chromosome, responsible for the fusion of part of the BCR (“breakpoint cluster region”) gene from chromosome 22 with the ABL gene on chromosome 9. This abnormal fusion gene generates a Bcr-Abl fusion protein carrying a tyrosine kinase domain capable of activating a cascade of proteins that control the cell cycle and speed up cell division.

Targeted therapies that specifically inhibit the activity of the Bcr-Abl protein can induce complete remissions in CML, confirming the central importance of bcr-abl as the cause of CML. Bcr-Abl specific inhibitors are tyrosine kinase inhibitors (TKIs) a pharmaceutical drug that inhibits tyrosine kinases. Tyrosine kinases are enzymes responsible for the activation of many proteins by signal transduction cascades. The proteins are activated by adding a phosphate group to the protein (phosphorylation), a step that TKIs inhibit.

While the outcomes of leukemia have improved in the developed world, with a five-year survival rate is 57% in the United States (in children under 15, the five-year survival rate is greater than 60% or even 90%, depending on the type of leukemia), and while in children with acute leukemia who are cancer-free after five years, the cancer is unlikely to return, many patients develop drug resistance. This is often the result of the acquisition of cancer cell mutations that render a targeted drug unable to bind to its target, and results in recurrence and/or progression of the disease, which is usually associated with unfavorable outcomes. Therefore, there is an unmeet need for new drugs for use in the treatment of leukemia, and specifically drug-resistant leukemia.

### SUMMARY OF THE INVENTION

The present invention is based on the seminal discovery NU6027 can be used to treat leukemia and drug-resistant leukemia by binding to a mutated ABL1 kinase domain with a greater K_{D} that known TKIs used for the treatment of leukemia.

In one embodiment, the present invention provides a method of treating leukemia in a subject including: administering to the subject a therapeutically effective amount of a cyclin dependent kinase (CDK) inhibitor, wherein the CDK inhibitor is NU6027, thereby treating leukemia in the subject.

In one aspect, the leukemia is resistant to one or more tyrosine kinase inhibitors (TKIs). In some aspects, the one or more TKIs are selected from the group consisting of imatinib, nilotinib, dasatinib, bosutinib, ibrutinib and ponatinib. In another aspect, the leukemia is characterized by a mutation in an ABL1 gene. In some aspects, the ABL1 mutation is T315I. In one aspect, the subject has previously been treated with imatinib, nilotinib, dasatinib, ibrutinib, ponatinub, or a combination thereof. In another aspect, the leukemia is selected from the group consisting of acute lymphoblastic leukemia (ALL), acute myeloid leukemia (AML), blastic plasmacytoid dendritic cell neoplasm (BPDCN), chronic lymphocytic leukemia (CLL), chronic myelogenous leukemia (CML), hairy cell leukemia, mast cell leukemia and meningeal leukemia. In some aspects, the leukemia is CML. In one aspect, NU6027 binds to a mutated ABL1 kinase domain in either a phosphorylated or unphosphorylated conformation. In some aspects, NU6027 binds to a mutated ABL1 kinase domain with a K_{D} that is at least 100 times greater that the K_{D} of imatinib, nilotinib, dasatinib, bosutinib, ibrutinib or ponatinib.

In another embodiment, the invention provides a method of treating a drug-resistant chronic myelogenous leukemia (CML) in a subject comprising: administering to the subject a therapeutically effective amount of NU6027, thereby treating the drug-resistant CML in the subject.

In one aspect, the CML is resistant to imatinib, nilotinib, dasatinib, bosutinib and/or ponatinib.

### BRIEF DESCRIPTION OF THE DRAWINGS

FIG. 1 is a vector map illustrating the construct used for the expression of the kinase domain (residues 229-499) of a mutant ABL1 protein including a T315I mutation.

FIG. 2 is a blot illustrating the efficient removal of the His tag attachment by the TEV protease.

FIG. 3 shows a Coomassie stained quantitative gel of partially purified and high purity unphosphorylated mutant ABL1_T315I kinase domain.

FIGS. 4A-4B illustrate the densitometry analysis of mutant ABL1_T315I kinase domain. FIG. 4A illustrates the densitometry analysis of 1 μg partially purified mutant ABL1_T315I kinase domain. FIG. 4B illustrates the densitometry analysis of 1 μg high purity mutant ABL1_T315I kinase domain.

FIGS. 5A-5B illustrate melt curves of tag removed mutant ABL1_T315I kinase domain. FIG. 5A illustrates melt curves of tag removed mutant ABL1_T315I kinase domain from TSA. FIG. 5B illustrates melt curves of tag removed mutant ABL1_T315I kinase domain from first derivative.

FIGS. 6A-6C illustrate the analysis of the phosphorylation status of mutant ABL1_T315I kinase domain by liquid chromatography-mass spectrometry (LC-MS). FIG. 6A illustrates the LC profile. FIG. 6B illustrates the LC profile with an electrospray ionization. FIG. 6C illustrates the deconvoluted spectra.

FIG. 7 shows an immunoblot of 10×-His-tev-ABL1(T315I)(299-449) illustrating the autophosphorylation ability of the kinase domain.

FIGS. 8A-8D illustrate sensorgrams of A-0001. FIG. 8A illustrates the sensorgram of A-0001 binding to the unphosphorylated form of ABL1(T315I)(299-449).

FIG. 8B illustrates the sensorgram of A-0001 binding to the unphosphorylated form of ABL1(T315I)(299-449) fitted using an equilibrium steady state model. FIG. 8C illustrates the sensorgram of A-0001 binding to the phosphorylated form of ABL1(T315I)(299-449).

FIG. 8D illustrates the sensorgram of A-0001 binding to the phosphorylated form of ABL1(T315I)(299-449) fitted using an equilibrium steady state model.

FIGS. 9A-9D illustrate sensorgrams of imatinib. FIG. 9A illustrates the sensorgram of imatinib binding to the unphosphorylated form of ABL1(T315I)(299-449).

FIG. 9B illustrates the sensorgram of imatinib binding to the unphosphorylated form of ABL1(T315I)(299-449) fitted using an equilibrium steady state model. FIG. 9C illustrates the sensorgram of imatinib binding to the phosphorylated form of ABL1(T315I)(299-449).

FIG. 9D illustrates the sensorgram of imatinib binding to the phosphorylated form of ABL1(T315I)(299-449) fitted using an equilibrium steady state model.

### DETAILED DESCRIPTION OF THE INVENTION

The present invention is based on the seminal discovery that NU6027 can be used to treat leukemia and drug-resistant leukemia by binding to a mutated ABL1 kinase domain with a greater K_{D} that known TKIs used for the treatment of suck leukemias.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of treating leukemia in a subject including: administering to the subject a therapeutically effective amount of a cyclin dependent kinase (CDK) inhibitor, wherein the CDK inhibitor is NU6027, thereby treating leukemia in the subject.

As used herein, the term “leukemia” refers to a group of cancer that develops in the early blood-forming cells. Clinically and pathologically, leukemia can be subdivided into a variety of large groups. The first division being between its acute and chronic forms. Acute leukemia is characterized by a rapid increase in the number of immature blood cells. The crowding that results from such cells makes the bone marrow unable to produce healthy blood cells resulting in low hemoglobin and low platelets. Immediate treatment is required in acute leukemia because of the rapid progression and accumulation of the malignant cells, which then spill over into the bloodstream and spread to other organs of the body. Acute forms of leukemia are the most common forms of leukemia in children. Chronic leukemia is characterized by the excessive buildup of relatively mature, but still abnormal, white blood cells. Typically taking months or years to progress, the cells are produced at a much higher rate than normal, resulting in many abnormal white blood cells. Whereas acute leukemia must be treated immediately, chronic forms are sometimes monitored for some time before treatment to ensure maximum effectiveness of therapy. Chronic leukemia mostly occurs in older people but can occur in any age group. Additionally, the diseases are subdivided according to which kind of blood cell is affected. This divides leukemias into lymphoblastic or lymphocytic leukemias and myeloid or myelogenous leukemias. In lymphoblastic or lymphocytic leukemias, the cancerous change takes place in a type of marrow cell that normally goes on to form lymphocytes, which are infection-fighting immune system cells. Most lymphocytic leukemias involve a specific subtype of lymphocyte, the B cell. In myeloid or myelogenous leukemias, the cancerous change takes place in a type of marrow cell that normally goes on to form red blood cells, some other types of white cells, and platelets. Combining these two classifications provides a total of four main categories. Within each of these main categories, there are typically several subcategories. Finally, some rarer types are usually considered to be outside of this classification scheme.

In some aspects, the leukemia is selected from the group consisting of acute lymphoblastic leukemia (ALL), acute myeloid leukemia (AML), blastic plasmacytoid dendritic cell neoplasm (BPDCN), chronic lymphocytic leukemia (CLL), chronic myelogenous leukemia (CML), hairy cell leukemia, mast cell leukemia and meningeal leukemia.

Acute lymphoblastic leukemia (ALL) is the most common type of leukemia in young children. It also affects adults, especially those 65 and older. Standard treatments involve chemotherapy and radiotherapy. Subtypes include precursor B acute lymphoblastic leukemia, precursor T acute lymphoblastic leukemia, Burkitt's leukemia, and acute biphenotypic leukemia. While most cases of ALL occur in children, 80% of deaths from ALL occur in adults.

Chronic lymphocytic leukemia (CLL) most often affects adults over the age of 55. It sometimes occurs in younger adults, but it almost never affects children. Two-thirds of affected people are men. The five-year survival rate is 85%. It is incurable, but there are many effective treatments. One subtype is B-cell prolymphocytic leukemia, a more aggressive disease.

Acute myelogenous leukemia (AML) occurs far more commonly in adults than in children, and more commonly in men than women. It is treated with chemotherapy. The five-year survival rate is 20%. Subtypes of AML include acute promyelocytic leukemia, acute myeloblastic leukemia, and acute megakaryoblastic leukemia.

Chronic myelogenous leukemia (CML) occurs mainly in adults; a very small number of children also develop this disease. It is treated with tyrosine kinase inhibitor such as imatinib or other drugs. The five-year survival rate is 90%. One subtype is chronic myelomonocytic leukemia.

Hairy cell leukemia (HCL) is sometimes considered a subset of chronic lymphocytic leukemia but does not fit neatly into this category. About 80% of affected people are adult men. No cases in children have been reported. HCL is incurable but easily treatable. Survival is 96% to 100% at ten years.

T-cell prolymphocytic leukemia (T-PLL) is a very rare and aggressive leukemia affecting adults; somewhat more men than women are diagnosed with this disease. Despite its overall rarity, it is the most common type of mature T cell leukemia; nearly all other leukemias involve B cells. It is difficult to treat, and the median survival is measured in months.

Large granular lymphocytic leukemia may involve either T-cells or NK cells; like hairy cell leukemia, which involves solely B cells, it is a rare and indolent (not aggressive) leukemia.

Adult T-cell leukemia is caused by human T-lymphotropic virus (HTLV), a virus similar to HIV. Like HIV, HTLV infects CD4+ T-cells and replicates within them; however, unlike HIV, it does not destroy them. Instead, HTLV “immortalizes” the infected T-cells, giving them the ability to proliferate abnormally. Human T-cell lymphotropic virus types I and II (HTLV-I/II) are endemic in certain areas of the world.

In some aspects, the leukemia is CML.

While the illustrative example in the present disclosure relates to treatment of leukemia, it should be understood that A-0001 can be used to treat other cancers related to ABL1 mutations. As further detailed below, ABL1 mutations are associated with uncontrolled proliferation in cells, which is a hallmark of cancer. ABL1 mutations have been described in a variety of cancer types, therefore invention drugs can inhibit mutated ABL1 kinase for the treatment of any such cancer. Accordingly, A-0001 can be used to treat subjects with cancers including hematological cancers (including leukemia, lymphomas, myelodysplastic syndromes and myeloproliferative neoplasms), as well as for lung, colon, endometrial, breast, prostate, bladder, ovarian, rectal, pancreatic, esophageal cancers, melanoma and glioblastoma, for example.

As used herein, “NU6027” can be referred to as “A-0001”, or “CAS 220036-08-8” without any difference in the meaning, and refers to the compound 6-Cyclohexylmethyloxy-5-nitroso-pyrimidine-2,4-diamine having the molecular formula C₁₁H₁₇N₅O₂, and the chemical formula

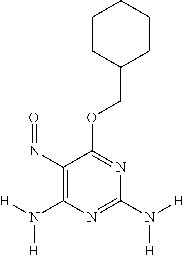 [SMILES: Nc1nc(N)c(N=O)c(OCC2CCCCC2)n1]

NU6027 is a selective cyclin-dependent kinase 2 (CDK2) inhibitor and a potent inhibitor of ATR signaling. NU6027 inhibits the growth of human tumor cells with mean GI50 of 10 μM. NU6027 causes a reduction cancer cell survival and proliferation by reducing the number of cells in S-phase (without affecting the number of cells in G1 or G2/M). NU6027 is a potent inhibitor of cellular ATR activity with IC50 of 6.7 μM in MCF7 cells and 2.8 μM in GM847KD cells and enhances hydroxyurea and cisplatin cytotoxicity in an ATR-dependent manner.

Cyclin-dependent kinase 2, also known as cell division protein kinase 2, or Cdk2, is an enzyme that in humans is encoded by the CDK2 gene, and a member of the cyclin-dependent kinase family of Ser/Thr protein kinases. Cdk2 is a catalytic subunit of the cyclin-dependent kinase complex, whose activity is restricted to the G1-S phase of the cell cycle, where cells make proteins necessary for mitosis and replicate their DNA. This protein associates with and is regulated by the regulatory subunits of the complex including cyclin E or A. Cyclin E binds G1 phase Cdk2, which is required for the transition from G1 to S phase while binding with Cyclin A is required to progress through the S phase. Its activity is also regulated by phosphorylation.

NU6027 can be administered alone or in combination with a pharmaceutically acceptable carrier. By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

Alternatively, a pharmaceutically acceptable salt of NU6027 can be administered. The term “pharmaceutically acceptable salts” refers to physiologically and pharmaceutically acceptable salts of the compounds of the invention, e.g., salts that retain the desired biological activity of the parent compound and do not impart undesired toxicological effects thereto.

As described herein, NU6027 is used for the treatment of leukemia and is therefore used for its protein tyrosine kinase inhibitory activity. Protein tyrosine kinase (PTK) is one of the major signaling enzymes in the process of cell signal transduction; it catalyzes the transfer of ATP-γ-phosphate to the tyrosine residues of the substrate protein (i.e., phosphorylating the protein), a process involved in the regulation of cell growth, differentiation, death and a series of physiological and biochemical processes. Abnormal expression of PTK usually leads to cell proliferation disorders, and is closely related to tumor invasion, metastasis and tumor angiogenesis. A variety of PTKs are used as targets in the screening of anti-tumor drugs. Tyrosine kinase inhibitors (TKIs) compete with ATP for the ATP binding site of PTK and reduce tyrosine kinase phosphorylation, thereby inhibiting cancer cell proliferation. The anti-tumor mechanism of TKI can be achieved by inhibiting the repair of tumor cells, blocking the cell division in G1 phase, inducing and maintaining apoptosis, anti-angiogenesis and so on.

TKIs are responsible for great progress in the treatment of cancer, but acquired resistance is inevitable, restricting the efficacy of the treatment of cancer. Even in highly sensitive patients with TKI, tumor cells can always self-adjust, look for a way out, to avoid TKI target, which ultimately can lead to acquired resistance and disease progression. As a result, most TKI therapies are only effective for a limited period of time.

Since their discovery, many TKIs having various PTK targets have been developed and approved for the treatment of cancer (Table 1).

TABLE 1
Non-exhaustive list of approved TKIs and their target(s)

| TKI | Target |
|---|---|
| Imatinib | Abl, PDGFR, SCFR |
| Gefitinib | EGFR |
| Nilotinib | Bcr-Abl, PDGFR |
| Sorafenib | Raf, VEGFR, PDGER |
| Sunitinib | PDGFR, VEGFR, |
| Dasatinib | Bcr-Abl, SRC, PDGFR |
| Lapatinib | EGFR |
| Pazopanib | VEGFR, PDGFR, FGFR |
| Crizotinib | ALK |
| Ruxolitinib | JAK1, JAK2 |
| vandetanib | VEGFR, EGFR |
| Axitinib | VEGFR |
| Bosutinib | Abl, SRC |
| Afatinib | EGFR |
| Erlotinib | EGFR |
| Ceritinib | ALK |
| Osimertinib | EGFR |
| Lenvatinib | VEGFR |
| Alectinib | ALK |
| Regorafenib | VEGFR, EGFR |
| Neratinib | HER2 |
| Brigatinib | ALK |
| Ibrutinib | BTK |
| Ponatinib | BCR-ABL |

In one aspect, the leukemia is resistant to one or more tyrosine kinase inhibitors (TKIs). In some aspects, the one or more TKIs are selected from the group consisting of imatinib, nilotinib, dasatinib, bosutinib, ibrutinib and ponatinib.

In most cases, there is no identified cause for leukemia. However, in some cases leukemia can be characterized by a genetic mutation leading to the uncontrolled proliferation of blood cells.

In some aspects, the leukemia is characterized by a mutation in an ABL1 gene.

Tyrosine-protein kinase ABL1 also known as ABL1 is a protein that, in humans, is encoded by the ABL1 gene located on chromosome 9. c-Abl is sometimes used to refer to the version of the gene found within the mammalian genome, while v-Abl refers to the viral gene, which was initially isolated from the Abelson murine leukemia virus. Mutations in the ABL1 gene are associated with chronic myelogenous leukemia (CML). In CML, the gene is activated by being translocated within the BCR (breakpoint cluster region) gene on chromosome 22. This new fusion gene, BCR-ABL, encodes an unregulated, cytoplasm-targeted tyrosine kinase that allows the cells to proliferate without being regulated by cytokines. This, in turn, allows the cell to become cancerous. This gene is a partner in a fusion gene with the BCR gene in the Philadelphia chromosome, a characteristic abnormality in chronic myelogenous leukemia (CML) and rarely in some other leukemia forms. The BCR-ABL transcript encodes a tyrosine kinase, which activates mediators of the cell cycle regulation system, leading to a clonal myeloproliferative disorder. The BCR-ABL protein can be inhibited by various small molecules.

Cancer cells are known to develop various mechanism to escape the efficacy of anti-cancer drug, including TKIs. Mutations in the kinase domain (KD) of BCR-ABL are the most prevalent mechanism of acquired resistance to TKIs in patients with chronic myeloid leukemia (CML). Specifically, some punctual mutation in the sequence of BCR-ABL have been shown to be associated with a resistance to TKIs such as imatinib.

In some aspects, the ABL1 mutation is T315I.

Drug-resistance can be innate (cancer cells are inherently resistant to the drug) or acquired (cancer cells become resistant to the drug after exposition to the drug). Acquired drug-resistance can be the result of the acquisition by the cancer cells of a drug-resistant mutation, which usually arises after exposure of a cancer cell to the drug and is a cancer mechanism to escape the mechanism of action of the drug. In various aspects, an acquired drug-resistance arise while or after the subject having cancer is treated with the drug.

In one aspect, the subject has previously been treated with imatinib, nilotinib, dasatinib, ibrutinib, ponatinub, or a combination thereof.

Drug-resistant mutations, such as ABL1 T315I mutation can induce change to the phosphorylation state of a protein, which in turn can impact the ability of a TKI to bind to a tyrosine kinase domain.

In one aspect, NU6027 binds to a mutated ABL1 kinase domain in either a phosphorylated or unphosphorylated conformation.

The efficacy of a drug, such as a TKI may be evaluated by a measure of the specific binding of the TKI to its target. As used herein, “specific binding” refers to TKI binding to a target tyrosine kinase domain (TKD). Typically, a TKI specific binding can be measured by a K_{D} of the TKI. The term “kd” (sec-1), as used herein, is intended to refer to the dissociation rate constant of a particular TKI/TKD interaction. This value is also referred to as the off value. The term “K_{D}” (M-1), as used herein, is intended to refer to the dissociation equilibrium constant of a particular TKI/TKD interaction.

Mutated TKDs that are resistant to TKI often present a reduced K_{D} as compared to the K_{D} or a non-mutated TKD, which explains the loss of efficacy of the TKI. NU6027, as described herein has a greater binding to mutated TKD than known TKIs.

In some aspects, NU6027 binds to a mutated ABL1 kinase domain with a K_{D} that is at least 100 times greater that the K_{D} of imatinib, nilotinib, dasatinib, bosutinib, ibrutinib or ponatinib.

For example, K_{D} of NU6027 for a mutated ABL1 kinase can be 10×, 20×, 30×, 40×, 50×, 60×, 70×, 80×, 90×, 100×, 150×, 200×, 250× or more times greater than the K_{D} of imatinib, nilotinib, dasatinib, bosutinib, ibrutinib or ponatinib for a mutated ABL1 kinase.

In another embodiment, the invention provides a method of treating a drug-resistant chronic myelogenous leukemia (CML) in a subject comprising administering to the subject a therapeutically effective amount of NU6027, thereby treating the drug-resistant CML in the subject.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of a leukemia or treatment of a drug-resistant leukemia). Such amount should be sufficient to treat leukemia in the subject. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In one aspect, the CML is resistant to imatinib, nilotinib, dasatinib, bosutinib and/or ponatinib.

Presented below are examples discussing the use of NU6027 contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used

### EXAMPLES

### Example 1

### Generation and Characterization of a Mutant ABL1(T315I) Kinase Domain

The goal of these experiments was to produce some tag-removed ABL1(T315I)(229-499) for crystallographic studies. In addition, protein was also produced during the course of the purifications that retained the 10×His-tag to enable studies by Surface Plasmon Resonance. The recombinant ABL1(T315I) (isoform 1a) residues 229-499 protein was designed with an N-terminal 10×His tag with a TEV protease cleavage sequence and was co-expressed with recombinant YopH phosphatase (to maintain the ABL1(T315I)(229-499) dephosphorylated form). The protein was also co-expressed with the pGro7 chaperone to improve the level of soluble protein expression. Biomass production and protein purification are based on Albanese et al. (2018, Biochemistry 57, 4675-4689) and Wilson et al. (2015, Science 347, 882-886) with minor modifications. Briefly, the protein purification consisted of TALON affinity chromatography, TEV cleavage, Ni-NTA, ion exchange chromatography and SEC. The resulting protein was concentrated and stored at −80° C. after flash-freeze in liquid nitrogen.

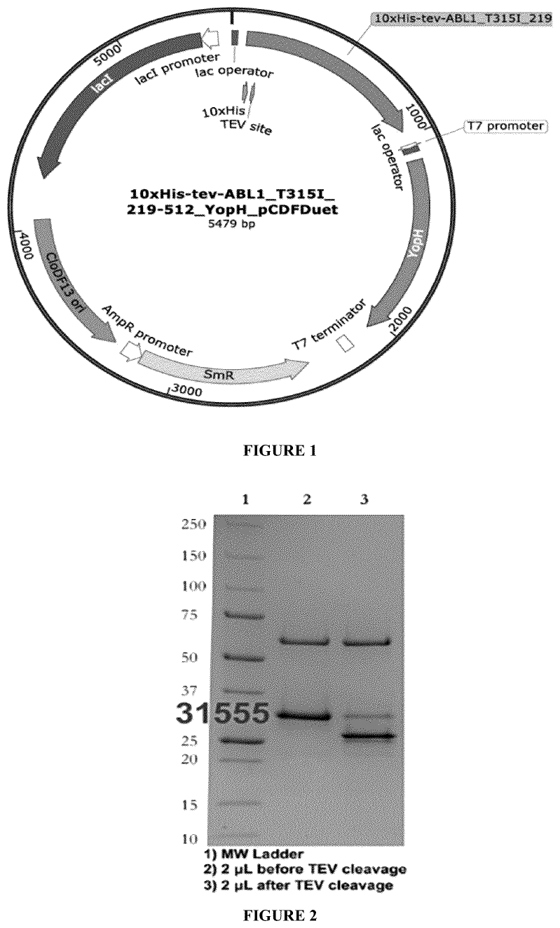

The ABL1(229-499)(T315I) kinase domain protein was produced through E. coli expression, with co-expressed with YopH phosphatase (to maintain dephosphorylated state) and a chaperone protein (to facilitate soluble expression) (see FIG. 1). As illustrated in FIG. 2, TEV cleavage was efficient at removing the His tag.

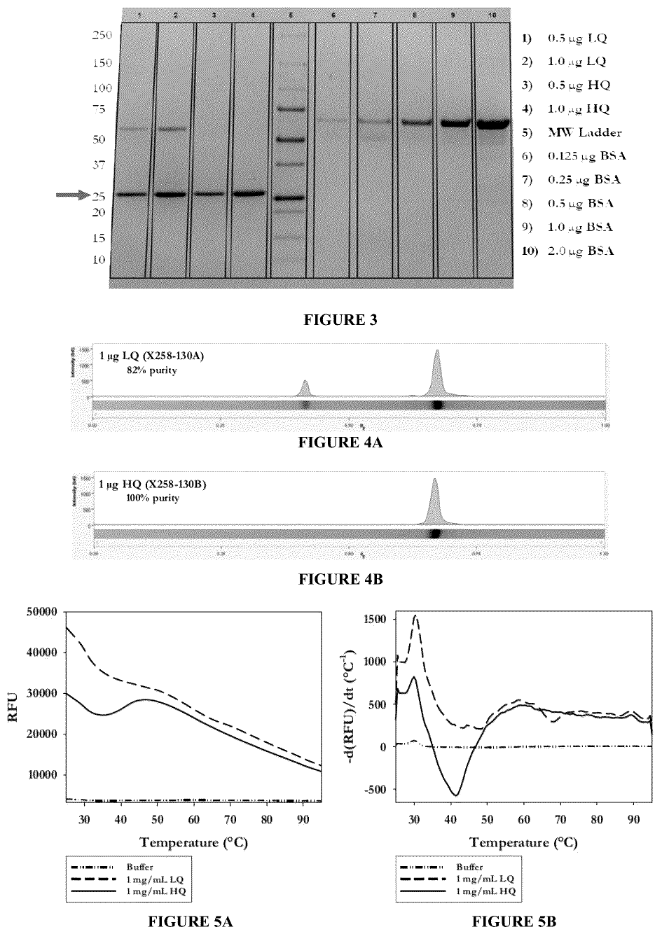

A total of five rounds of protein expression and purification have been undertaken to optimize the methods and generate mg quantities of highly pure ABL1(229-499)(T315I) protein, both in the unphosphorylated and as the phosphorylated form. This provided the means to optimize the various stages of the process and to increase yields. The last round of purification involved 11 Liters of cell culture with and overnight induction at 18° C. As illustrated in FIG. 3, the purification of the kinase domains was assessed in a Coomassie 4-20% gradient gel in reducing conditions. In lanes 1 and 2 partially purified ABL1(T315)(229-449), LQ (lower quality) was obtained by removing the tag and was found 82% pure by densitometry (see FIG. 4A), as it retained some of the pGro7 chaperone. The 3.8 mg amount was stored for future repurification. As shown in lanes 3 and 4 high purity unphosphorylated ABL1(T315)(229-449), HQ (high quality) was obtained by removing the tag and was found 100% pure by densitometry (see FIG. 4B). A total of 6.0 mg of pure protein was stored in aliquots for using in the biophysical studies. Yield of 0.55 mg/L culture. Lanes 6-10 includes BSA standards.

As shown in Table 2, and in FIG. 5, the purity of the protein impacted the melting temperature.

TABLE 2
Melting temperatures of tag removed ABL1(T315I)(229-499).

| Construct | Tm (° C.) |
|---|---|
| Buffer | / |
| LQ: 1 mg/mL | very weak signal, 42-48 |
| HQ: 1 mg/mL | 41.5 |

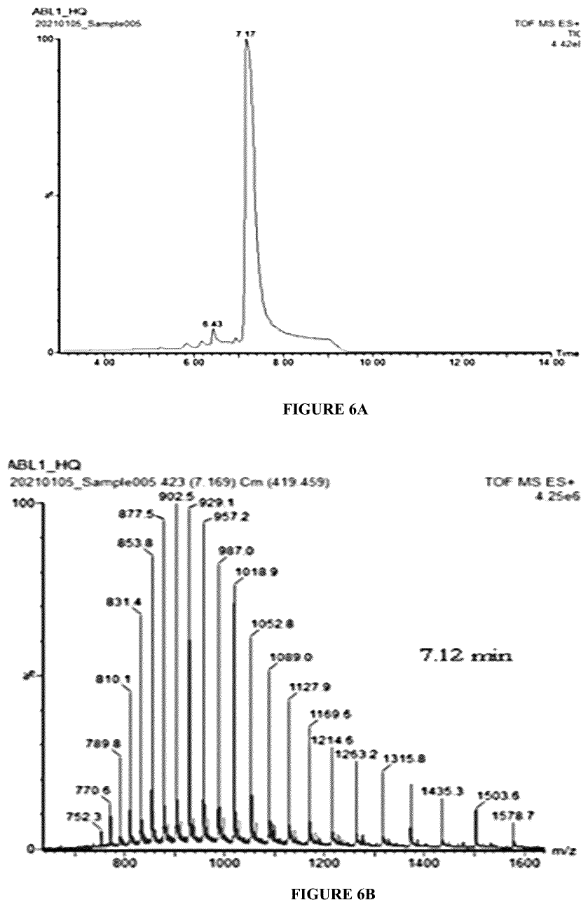

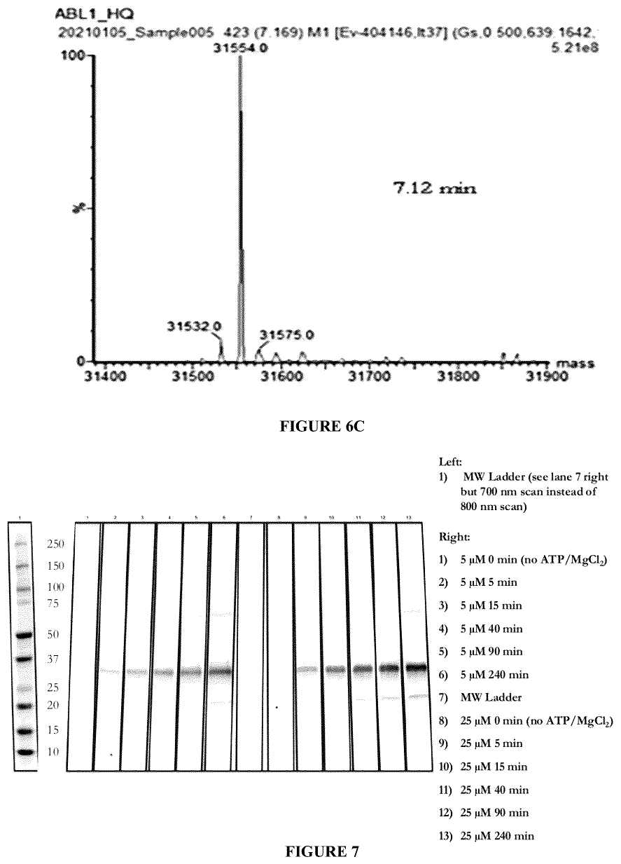

The phosphorylation state of the final protein was checked by western blot using anti-phosphor-Tyr393 Ab (Abcam ab4717). ABL1 was found completely dephosphorylated. As illustrated in FIGS. 6A-6C, this was further confirmed by LC-MS with electrospray ionization, which clearly demonstrated the protein was in the unphosphorylated state by yielding a measured molecular weight is 31,554 daltons for a calculated molecular weight of 31,555 daltons.

The activity of ABL1(T315I)(229-499) as an active kinase was demonstrated by an autophosphorylation reaction when ATP/MgCl2 was added to the purified ABL1(T315I)(229-499) and incubated (see FIG. 7). The protocol was adapted to generate phosphorylated 10×His-tev-ABL1(T315I)(229-499) for use in the SPR studies. Briefly, ABL1 was completely dephosphorylated prior to the addition of ATP/MgCl2, ATP/MgCl2 was added to initiate autophosphorylation of ABL1. The amount of phosphorylated kinase increased over time indicating that ABL1 was active. It was noted that autophosphorylation of the 25 μM sample was faster than autophosphorylation of the 5 μM sample as autophosphorylation is an intermolecular reaction and therefore was concentration dependent.

### Example 2

### Evaluation of A-0001 Binding to Mutant ABL1(T315I) Kinase Domain

The Surface Plasmon Resonance (SPR) analysis was developed for measuring A-0001 compound binding to the unphosphorylated and to the phosphorylated forms of ABL1(229-499)(T315I).

Surface plasmon resonance required the attachment of the ligand to a surface and then flowing the analytes across this surface to measure the kinetic rate of the association (K_{A}) and the of kinetic rate dissociation (K_{D}) for a ligand interacting with the immobilized protein. From this measurement the equilibrium binding constant (K_{D}) is calculated. The SPR method was developed to enable the testing of compound binding against both the unphosphorylated and phosphorylated forms of 10×His-tev-ABL1(T315I)(229-499).

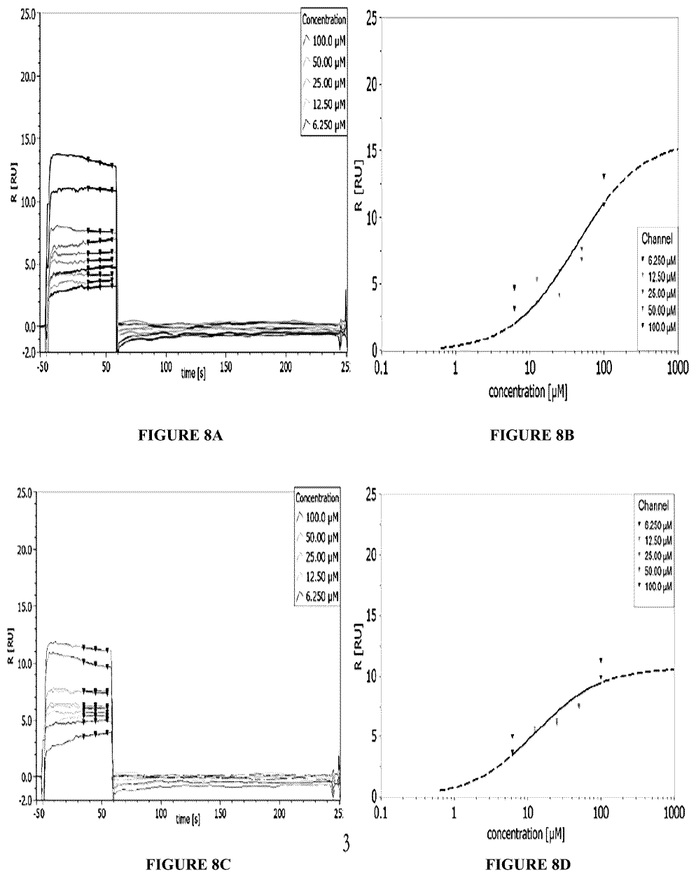

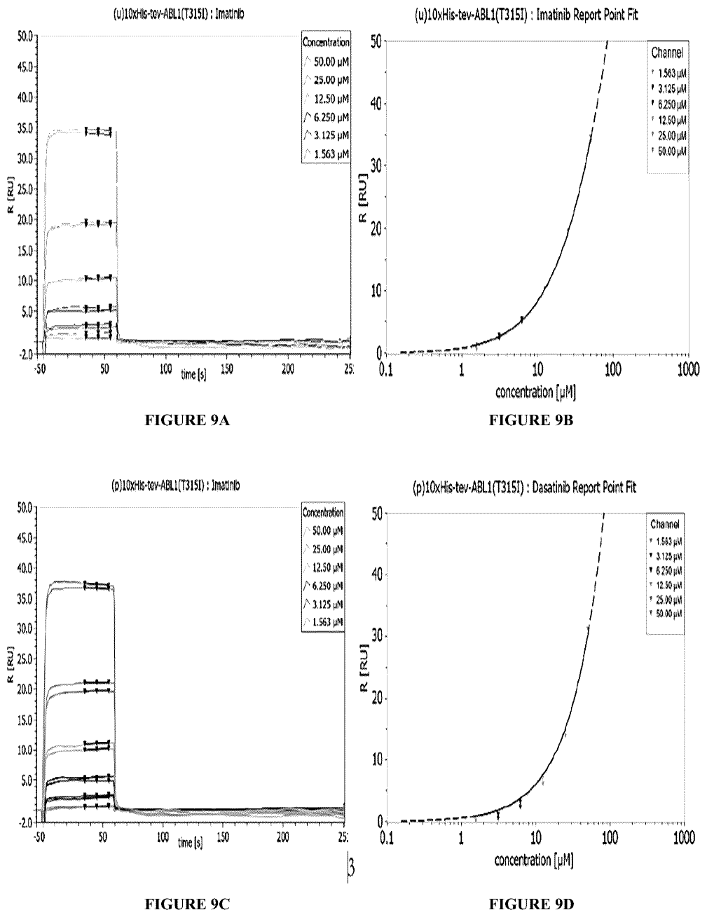

SPR binding measurements have been performed for A-0001. The SPR measurements were performed in two different buffer systems to evaluate potential buffer effects (HEPES vs Phosphate) to develop the methods and replicate the results. It was also confirmed that the ABL1(T315I) does not significantly bind (>100 μM) to Imatinib (approved for Pt line therapy) and Dasatinib (approved for 2^{nd} line therapy), demonstrating the drug-resistant affect that the T315I mutation has (see Table 3 and FIGS. 8A-8D and 9A-9D).

TABLE 3
Summary of current SPR results for compounds binding to the unphosphorylated or
phosphorylated ABL1(T315I)(229-499) proteins. Attachment to the biosensor surface
was through Ni-NTA capture of the 10xHis-tag present on the N-terminus.

| | | Unphosphorylated | Phosphorylated | Unphosphorylated | Phosphorylated |
|---|---|---|---|---|---|
| | | ABL1(T315I) | ABL1(T315I) | ABL1(T315I) | ABL1(T315I) |
| | | (229-499) in | (229-499) in | (229-499) in | (229-499) in |
| Scaffold | MW | HBST buffer | HBST buffer | PBST buffer | PBST buffer |
| ID-# | (daltons) | KD (μM) | KD (μM) | KD (μM) | KD (μM) |
| A-0001 | 251 | 2 μM | 13 μM | 5 μM | 16 μM |
| Dasatinib | 488 | >100 | >100 | >100 | >100 |
| Imatinib | 494 | >100 | >100 | >100 | >100 |
| DCC2036 | 553 | >100 | >100 | >100 | >100 |

### Example 3

### Evaluation of the ABL1 Inhibition of A-0001

To assess inhibitory effects of A-0001 on cancer cell resistant to tyrosine kinase inhibitor such as dasatinib or imatinib, cell-based assays using patient derived cell lines will be established. The patients will be selected for having a T315I ABL1 mutation, and a drug resistant disease.

Upon confirmation of the in vitro efficacy of A-0001, preclinical evaluations will be performed. Standard SAR assessments, structure-guided drug design, and scale ups for characterization of drug-like properties and animal studies will be implemented. The specificity of the ligands for the target kinase and related kinases will be evaluated. As is standard practice with kinase inhibitors, panels of kinases will need to be assessed to confirm the relative specificity of the key compounds. Further cell-based assays to assess and confirm target engagement with the cells and to validate the mechanism of cell death will be performed. Pharmacological parameters (ADME and DMPK), and pharmacokinetic properties will be evaluated. Animal model will be developed to assess in vivo tolerability, and efficacy.

Although the invention has been described with reference to the above examples, it will be understood that modifications and variations are encompassed within the spirit and scope of the invention. Accordingly, the invention is limited only by the following claims.

What is claimed is:

1. A method of treating leukemia in a subject comprising administering to the subject a therapeutically effective amount of a cyclin dependent kinase (CDK) inhibitor, wherein the CDK inhibitor is NU6027,
thereby treating leukemia in the subject.

2. The method of claim 1, wherein the leukemia is resistant to one or more tyrosine kinase inhibitors (TKIs).

3. The method of claim 2, wherein the one or more TKIs are selected from the group consisting of imatinib, nilotinib, dasatinib, bosutinib, ibrutinib and ponatinib.

4. The method of claim 1, wherein the leukemia is characterized by a mutation in an ABL1 gene.

5. The method of claim 4, wherein the ABL1 mutation is T315I.

6. The method of claim 1, wherein the subject has previously been treated with imatinib, nilotinib, dasatinib, ibrutinib, ponatinub, or a combination thereof.

7. The method of claim 1, wherein the leukemia is selected from the group consisting of acute lymphoblastic leukemia (ALL), acute myeloid leukemia (AML), blastic plasmacytoid dendritic cell neoplasm (BPDCN), chronic lymphocytic leukemia (CLL), chronic myelogenous leukemia (CML), hairy cell leukemia, mast cell leukemia and meningeal leukemia.

8. The method of claim 1, wherein the leukemia is CML.

9. The method of claim 1, wherein NU6027 binds to a mutated ABL1 kinase domain in either a phosphorylated or unphosphorylated conformation.

10. The method of claim 1, wherein NU6027 binds to a mutated ABL1 kinase domain with a K_{D} that is at least 100 times greater that the K_{D} of imatinib, nilotinib, dasatinib, bosutinib, ibrutinib or ponatinib.

11. A method of treating a drug-resistant chronic myelogenous leukemia (CML) in a subject comprising administering to the subject a therapeutically effective amount of NU6027, thereby treating the drug-resistant CML in the subject.

12. The method of claim 11, wherein the CML is resistant to imatinib, nilotinib, dasatinib, bosutinib and/or ponatinib.
